(19) United States
(12) Patent Application Publication
(10) Pub. No.: US 2008/0241270 A1
(43) Pub. Date: Oct. 2, 2008

(54) FLUID COMPOSITION FOR INHIBITING SURGICAL ADHESION FORMATION AND RELATED METHOD OF PRODUCTION

(76) Inventors: Robert A. Neal, Wales, ME (US); Robert B. Ray, Manchester, ME (US)

Correspondence Address: CHRIS A. CASEIRO, VERRILL DANA, LLP, ONE PORTLAND SQUARE, PORTLAND, ME, 04112-0586, US

(21) Appl. No.: 11/693,994

(22) Filed: Mar. 30, 2007

(51) Int. Cl. A61K 33/42; A61P 41/00

(52) U.S. Cl. 424/601

(57) ABSTRACT

A composition to inhibit the formation of adhesions that may arise at a surgical site. The composition includes an antioxidant in a carrier. The carrier is comprised of cocamide diethanolamine, diethanolamine and glycerin. The composition may include additional additives, such as euricamide and a compound including a polyethylene resin, calcium carbonate and a concentrated form of the carrier. The composition is produced by heating the carrier to a temperature, preferably about 375° F. and then adding the additives. The mixture is heated at temperature long enough to cause the antioxidant to leach into the liquid. The mixture is then cooled at room temperature and maintained in liquid form for ease of application to the surgical site where adhesion formation inhibition is desired. The composition is preferably degradable in vivo.

### BACKGROUND OF THE INVENTION

1. Field of the Invention

The present invention relates to treatments intended to inhibit the development of post-surgery adhesions. More particularly, the present invention relates to a fluid composition including a polymeric compound precursor and an antioxidant, and the method for creating the fluid.

2. Description of the Prior Art

Adhesions are a common complication of surgery. They may develop in a variety of areas in the body, and are characterized in that tissues which were separate before surgery become bonded together in the process of healing. The type and degree of damage caused by adhesions is variable, ranging from life-threatening, as in the intestines due to blockage, to extremely disabling, as in tendons or spinal cord, to chronic pain and infertility in the pelvic cavity, to being obstructive of further surgery in the pericardium. Postoperative formation of pelvic adhesions remains a serious problem in patients undergoing gynecological surgery.

Injury to intact peritoneum as a result of surgical insult or infection begins a cascade of undesired events. Within hours of surgical insult or infection, there is damage to arteries and veins. The resultant fluid emissions and initial fibrin development lead to fibrin matrix adhesions, as well as fiber growth and expansion. This development further leads to inflammation and the accompanying cell migration to the site of inflammation. Associated with this cell migration is a rapid respiratory burst leading to the generation of oxygen radicals at the site of inflammation. In the absence of sufficient free radical scavengers, high concentrations of oxygen radicals are capable of damaging the surrounding intact cells, including those responsible for vascular integrity. The increased permeability of blood vessels can lead to exudation of fluids that become the source for further adhesion matrix development. In addition, increased vascular permeability leads to local interruption of blood flow and eventual cell death in the vessels comprising the vascular supply. Reperfusion of tissues, following this ischemic event, leads to further generation of oxygen radicals and, ultimately, further exacerbates the degree of adhesion formation.

Under normal circumstances, the body can absorb these fibrinous deposits and sustains conventional healing. However, in the presence of severe tissue injury (e.g. following surgical trauma), the body is not always able to absorb the deposits, leading to abnormally persistent fibrin deposits and, ultimately, mature collagenous adhesions. Meticulous manual removal of the adhesions through one or more surgical procedures continues to be the most widely accepted treatment for existing adhesions. A substantial fraction of surgery therefore requires follow-up surgery to repair the effects of the adhesions caused by the initial surgery. This is not a desired outcome of any surgical procedure.

Many attempts have been made to address the issue of post-surgery adhesion formation. They include infusion of liquids into the pelvic cavity at the time of surgery, mechanical barriers between two opposing surfaces, and intravenously injected or topically applied pharmacologic agents. An example of such a commercially available product is the Seprafilm® adhesion barrier available from Genzyme Corporation of Cambridge, Mass. However, the present inventors are not aware of any method or product currently available that effectively resolves the problem and, as a result, the incidence of symptomatic adhesion formation remains high, and the clinical need for adhesion prevention still exists.

The therapies that have been attempted with some vague indications of potential success have proven not to be effective in preventing the initial formation of primary adhesions. For example, lavages with some forms of water-soluble polymers and/or biologically active molecules have not been very effective. Permanent mechanical barriers, such as Teflon™ sheets, can be effective but are difficult to remove and are subject to shifting at the site of adhesion formation. Degradable barriers such as oxidized cellulose, available under the name InterCeed™ from the Johnson & Johnson company, and specific degradable polymeric gels may have some utility but do not completely address the problem in all instances. A detailed discussion of this background information is provided in U.S. Pat. No. 6,780,427, the content of which is incorporated herein by reference.

The mechanism behind the limited success of those treatments having at least some success appears to be centered on the inhibition of the pathway leading to oxidative damage to tissue. That is, the types of treatments that include the capability to destroy active oxygen species, such as superoxide, peroxide, hydrogen peroxide or hydroxyl radicals. Again, however, these active oxygen inhibitors have not met with enough success to be widely accepted in the operating room. Moreover, they appear to have limited value in preventing re-adhesion after primary adhesion removal.

Initial studies conducted by John P. Delaney, M.D. Ph.D., FACS at the University of Minnesota Medical School, Department of Surgery, Minneapolis, Minn., of an antistatic film available from Pure-Stat™ Technologies, Inc., of Lewiston, Me., and assignee of the present invention, indicated that this particular film had advantageous adhesion formation inhibition properties. The evaluations involved performing surgical procedures on test rats in which it was a goal to cause the formation of adhesions. While Dr. Delaney's evaluation showed that the film was promising, it was determined that the film itself was not suitable for the intended purpose as it would require the application of many square inches of the film to provide effective inhibition in large areas, such as the abdomen. Further, the film would likely shift after placement and would therefore either be absent where needed or could cause separate problems upon migration and/or bunching. Dr. Delaney indicated that a liquid having adhesion formation inhibition characteristics as exhibited by the Pure-Stat™ film would be more desirable. Therefore, it was a goal of the inventors to develop a liquid composition exhibiting suitable effectiveness in adhesion formation inhibition.

Therefore, what is needed is an effective composition in liquid form for preventing adhesion formation. Further, what is needed is such a composition that may be integrated into the surgical procedure that would otherwise result in the generation of the adhesions. Yet further, what is needed is a method for making such a composition.

### SUMMARY OF THE INVENTION

It is an object of the present invention to provide an effective composition for preventing adhesion formation. It is also an object of the present invention to provide such a composition that may be integrated into the surgical procedure that would otherwise result in the generation of the adhesions. Further, it is an object of the present invention to provide a method for making such a composition.

These and other objectives are achieved in the present invention, which is a composition comprising one or more antioxidants in a Cocamide diethanolamine (DEA) carrier. The composition optionally includes one or more other additives but at least includes the antioxidant and the carrier. The carrier, in liquid form, is heated to a high temperature and the antioxidant, in a solid form, is added to the carrier and heated at temperature until melted. The liquid combination has been found through tests to produce an effective adhesion formation inhibitor. The liquid composition is preferably degradable in vivo.

The invention further includes a method of producing the composition including the steps of heating the carrier to a selected temperature at least sufficient to melt any solid components, including to about 375° F., adding the additives until at least leaching into the liquid carrier the antioxidant, and then cooling the liquid to room temperature while maintaining the liquid in liquid form at room temperature.

### DETAILED DESCRIPTION OF THE INVENTION

The present invention is a composition that is an effective post-surgery adhesion formation inhibitor that is degradable in vivo. The composition includes one or more antioxidants in a carrier. The carrier may be a combination of a coconut fatty acid DEA, diethanolamine and glycerin. The composition may also include a filler such as calcium carbonate. An antioxidant determined to be suitable for the purpose of the present invention is a compound blend of about 67% by weight of a phosphite (such as an Irgafos® product such as Irgafos® 168 available from Ciba Specialty Chemicals of Basel, Switzerland) or a thioester, and about 33% by weight of a phenolic antioxidant compound (such as an Irganox® product such as Irganox® 110 also available from Ciba Specialty Chemicals). Alternative antioxidants suitable for this purpose include hydroxylamine, lactone and vitamin E. The components of the composition are combined together in a variety of combinations, with examples showing efficaciousness described hereinbelow. The coconut fatty acid DEA, diethanolamine, glycerin may be found in satisfactory proportions in the product identified as Alkamide™ CDD-518 available from Rhodia Inc. of Cranbury, N.J.

An aspect of the invention includes the method of preparing the composition described above. Specifically, it has been determined that it is necessary to heat the composition to a temperature of at least 375° F. for at least 15 minutes. The heated composition may then be cooled down and maintained in liquid form.

The composition of the present invention created through the production process described herein is completely effective in preventing post-surgery adhesion formation in preliminary studies conducted.

The antistatic film that exhibited desired adhesion inhibition characteristics included the Alkamide™ CDD-518 liquid antistatic compound concentrated into a pellet in a polyethylene resin including a slip agent, such as euricamid wax, an antioxidant and a silica used to prevent the film from sticking or blocking to itself. The pellets containing the concentrated antistatic compound is ordinarily extruded into film form at elevated temperatures and pressures. Initial evaluations led to the understanding that neither the antistatic compound in liquid form, nor the resin pellets containing the antistatic compound, could provide adhesion inhibition. Instead, it was only the prepared film that exhibited the desired characteristic. As a result, the inventors set out to resolve the suitable combination of components and process steps required to produce a liquid form of composition having effective adhesion inhibition properties.

The inventors determined that the particular combination of components identified herein and processed in the manner described produced an effective adhesion inhibitor. This discovery was made based upon the evaluation of six samples prepared in accordance with the steps herein described. The evaluation was conducted by Dr. Delaney in accordance with a procedure similar to the one he used in initially evaluating the Pure-Stat™ film product described herein. The evaluations involved performing surgical procedures on test rats in which it was a goal to cause the formation of adhesions. The compositions described herein were applied in liquid form to the locations where the surgical procedures were performed and the subjects examined seven days after the operations were performed. The first three composition samples described herein comprised only the antistatic compound under different processing conditions.

Sample A

Sample A was a liquid sample of the Alkamide™ CDD-518 compound heated to 325° F. for 10 minutes, cooled and transferred in liquid form at room temperature for in vivo test.

Sample B

Sample B was a liquid sample of the Alkamide™ CDD-518 compound heated to 350° F. for 20 minutes, cooled and transferred in liquid form at room temperature for in vivo test.

Sample C

Sample C was a liquid sample of the Alkamide™ CDD-518 compound heated to 375° F. for 30 minutes, cooled and transferred in liquid form at room temperature for in vivo test.

Samples A, B and C all failed to exhibit effective adhesion prevention properties. It was therefore determined that the antistatic compound alone was not the active composition for adhesion formation inhibition.

Thereafter, the antistatic compound was combined with additional additives that formed part of the antistatic film that had been identified as effective in preventing adhesion formation. These compounds were prepared and evaluated in the following three samples.

Sample 1

Sample 1 contained Alkamide™ CDD-518 and euricamide in the following combination: three pounds of Alkamide™ CDD-518 and 0.15 pounds of the euricamide, for an antistatic to euricamide weight proportion of about 95% Alkamide and about 5% euricamide. In process, the Alkamide™ CDD-518 alone was heated to 375° F. and, upon reaching that temperature, the euricamide was added. The combination was mixed at temperature until the euricamide pellets melted and leached into and combined with the hot liquid Alkamide™ CDD-518. The combination was then cooled and transferred in liquid form at room temperature for in vivo test. The euricamide was obtained from the Ampacet Corporation of Tarrytown, N.Y., in concentrated polyethylene-based pellets.

Sample 2

Sample 2 contained Alkamide™ CDD-518, an antioxidant compound identified as a blend of about 67% by weight of the Irgafos® 168 phosphite and about 33% by weight of the Irganox® 110 phenolic components previously described herein in pellet form, and a compound identified as Pink Purestat™ available from Pure-Stat™ Technologies, Inc., of Lewiston, Me., comprised of polyethylene resin, a calcium carbonate filler, and concentrated Alkamide™ CDD-518 available combined together in pellet form in that commercially available product. All three components joined in the following proportions: three pounds of Alkamide™ CDD-518, 0.6 pounds of the antioxidant, and 0.15 pounds of the Pink Purestat™, for an antistatic to antioxidant to Pink Purestat™ weight proportion of about 80% Alkamide™ CDD-518, about 16% antioxidant, and about 4% Pink Purestat™. In process, the Alkamide™ CDD-518 alone was heated to 375° F. and, upon reaching that temperature, the antioxidant and Pink Purestat™ were added. The combination was mixed at temperature until the additive pellets melted and the antioxidant and the calcium carbonate from the Pink Purestat™ leached into and combined with the liquid Alkamide™ CDD-518. The combination was then cooled and transferred in liquid form at room temperature for in vivo test. The antioxidant was obtained from the Ampacet Corporation of Tarrytown, N.Y., in concentrated polyethylene-based pellets.

Sample 3

Sample 3 contained Alkamide™ CDD-518, the euricamide, the antioxidant, and the Pink Purestat™ in the following combination: three pounds of Alkamide™ CDD-518, 0.15 pounds of euricamide, 0.6 pounds of the antioxidant, and 0.15 pounds of the Pink Purestat™, for the following weight proportions: about 76% Alkamide™ CDD-518, about 4% euricamide, about 15% antioxidant, and about 4% Pink Purestat™. In process, the Alkamide™ CDD-518 alone was heated to 375° F. and, upon reaching that temperature, the euricamide, antioxidant and Pink Purestat™ were added in pellet form. The combination was mixed at temperature until the additive pellets melted and the antioxidant and the calcium carbonate from the Pink Purestat™ leached into and combined with the liquid Alkamide™ CDD-518. The combination was then cooled and transferred in liquid form at room temperature for in vivo test.

Sample 1 was not tested for adhesion formation inhibition; however, Samples 2 and 3 were, and both exhibited effective adhesion formation inhibition.

It is to be understood that the compositions and related processes described herein are but representations of options for the adhesion formation inhibition system of the present invention. This description is not intended to limit the principle concept of the present invention and the present invention includes equivalents thereto as established through the following appended claims.

What is claimed is:

1. A composition to inhibit the formation of adhesions at a surgical site, the composition comprising in liquid form at room temperature an effective amount of an antioxidant in an antistatic carrier, wherein the antistatic carrier is a compound comprised of coconut fatty acid diethanolamine, diethanolamine and glycerin.

2. The composition of claim 1 wherein the composition is degradable in vivo.

3. The composition of claim 1 wherein the carrier comprises about 76% to about 95% by weight of the composition and the antioxidant comprises about 15% by weight of the composition.

4. The composition of claim 1 further comprising euricamide.

5. The composition of claim 4 wherein the carrier comprises about 76% to about 95% by weight of the composition, the antioxidant comprises about 15% by weight of the composition, and the euricamide comprises about 5% by weight of the composition.

6. The composition of claim 1 further comprising a compound including a polyethylene resin, calcium carbonate and a concentrated form of the carrier.

7. The composition of claim 6 wherein the carrier comprises about 76% to about 95% by weight of the composition, the antioxidant comprises about 15% by weight of the composition, and the compound comprises about 4% by weight of the composition.

8. The composition of claim 1 further comprising euricamide and a compound including a polyethylene resin, calcium carbonate and a concentrated form of the carrier.

9. The composition of claim 8 wherein the carrier comprises about 76% to about 95% by weight of the composition, the antioxidant comprises about 15% by weight of the composition, the euricamide comprises about 5% by weight of the euricamide and the compound comprises about 4% by weight of the composition.

10. The composition of claim 1 wherein the antioxidant is a combination of a phosphite and a phenol, wherein the phosphite is about 67% by weight of the combination and the phenol is about 33% by weight of the combination.

11. A method for producing a composition for inhibiting the formation of adhesions at a surgical site, the composition including an antioxidant, an antistatic carrier and a compound including in solid form a polyethylene resin, calcium carbonate and a concentrated form of the carrier, wherein the antistatic carrier is a compound comprised of coconut fatty acid diethanolamine, diethanolamine and glycerin, the method comprising the steps of:
a. placing the antistatic carrier in a container;
b. heating the antistatic carrier to about 375° F.;
c. adding the antioxidant and the compound to the container including the heated antistatic carrier;
d. mixing the antistatic carrier, the antioxidant and the compound together;
e. maintain heating of the mixture until the antioxidant and the calcium carbonate of the compound leach into the antistatic carrier; and
f. cooling the mixture to room temperature in liquid form after the mixture is substantially completely in liquid form.

12. The method of claim 11 wherein the carrier comprises about 76% to about 95% by weight of the composition, the antioxidant comprises about 15% by weight of the composition, and the compound comprises about 4% by weight of the composition.

13. The method of claim 11 wherein the antioxidant is a combination of a phosphite and a phenol, wherein the phosphite is about 67% by weight of the combination and the phenol is about 33% by weight of the combination.

14. A method for producing a composition for inhibiting the formation of adhesions at a surgical site, the composition including an antioxidant, an antistatic carrier, euricamide and a compound including in solid form a polyethylene resin, calcium carbonate and a concentrated form of the carrier, wherein the antistatic carrier is a compound comprised of coconut fatty acid diethanolamine, diethanolamine and glycerin, the method comprising the steps of:
a. placing the antistatic carrier in a container;
b. heating the antistatic carrier to about 375° F.;
c. adding the antioxidant, the euricamide and the compound to the container including the heated antistatic carrier;
d. mixing the antistatic carrier, the antioxidant, the euricamide and the compound together;
e. maintain heating of the mixture until the antioxidant and the calcium carbonate of the compound leach into the antistatic carrier; and
f. cooling the mixture to room temperature in liquid form after the mixture is substantially completely in liquid form.

15. The method of claim 14 wherein the carrier comprises about 76% to about 95% by weight of the composition, the antioxidant comprises about 15% by weight of the composition, the euricamide comprises about 5% by weight of the euricamide and the compound comprises about 4% by weight of the composition.

16. The method of claim 14 wherein the antioxidant is a combination of a phosphite and a phenol, wherein the phosphite is about 67% by weight of the combination and the phenol is about 33% by weight of the combination.
